(19) United States
(12) Patent Application Publication
(10) Pub. No.: US 2025/0303098 A1
(43) Pub. Date: Oct. 2, 2025

(54) HOLDER FOR ATTACHING A GAS SENSOR TO AND/OR IN A HOUSING OF A VENTILATOR

(71) Applicant: Loewenstein Medical Technology S.A., Luxembourg (LU)

(72) Inventor: Fritz MEHR, Merenberg (DE)

(21) Appl. No.: 19/095,598

(22) Filed: Mar. 31, 2025

(30) Foreign Application Priority Data
Apr. 2, 2024 (DE) 10 2024 109 225.4

(51) Int. Cl. A61M 16/08; A61M 16/00; A61M 39/10

(52) U.S. Cl. CPC A61M 16/0816; A61M 16/0003; A61M 39/105; A61M 2202/0007; A61M 2202/02; A61M 2205/0216; A61M 2205/3327; A61M 2209/08

(57) ABSTRACT

A holder for attaching a gas sensor to and/or in a housing of a ventilator which comprises a base body having an attachment section for attaching the base body to and/or in the housing, a gas inlet, a gas outlet, a first receptacle for accommodating a coupling device such that an exit opening in the coupling device is fluidically coupled to the gas inlet, and a second receptacle for accommodating the gas sensor, the base body delimiting, together with the gas sensor accommodated by the second receptacle, a breathing gas chamber connected to the gas inlet and the gas outlet such that when the coupling device is accommodated by the first receptacle, breathing gas can flow from the exit opening via the gas inlet into the breathing gas chamber and from there via the gas outlet into an environment of the base body.

### CROSS-REFERENCE TO RELATED APPLICATIONS

The present application claims priority under 35 U.S.C. § 119 of German Patent Application No. 102024109225.4, filed Apr. 2, 2024, the entire disclosure of which is expressly incorporated by reference herein.

### BACKGROUND OF THE INVENTION

### 1. Field of the Invention

The invention relates to a holder for attaching a gas sensor to and/or in a housing of a ventilator. The invention further relates to a ventilator having such a holder.

### 2. Discussion of Background Information

A ventilator is usually equipped with what is known as an oxygen calibration module to determine an oxygen concentration in the breathing gas flow. For maintenance, cleaning, or repair purposes, the oxygen calibration module should be easily removable, in particular without needing to remove the housing of the ventilator.

In view of the foregoing, it would be advantageous to have available a holder that allows improved mounting, and/or disassembly, of a gas sensor on and/or in a ventilator and a correspondingly improved ventilator.

### SUMMARY OF THE INVENTION

In a first aspect, the invention provides a holder for attaching a gas sensor to and/or in a housing of a ventilator. The ventilator may comprise, in addition to the gas sensor and the housing, a breathing gas connector for connecting a breathing gas source, a patient connector for connecting a patient interface, and a coupling device for fluidically coupling the gas sensor to the breathing gas connector and/or the patient connector. The holder comprises a base body with an attachment section for attaching the base body to and/or in the housing, a gas inlet, a gas outlet, a first receptacle for accommodating the coupling device such that an exit opening of the coupling device is fluidically coupled to the gas inlet, and a second receptacle (for example arranged next to the first receptacle) for accommodating the gas sensor. The basic body is formed in such a way that it delimits, together with the gas sensor accommodated by the second receptacle, a breathing gas chamber, which is connected to the gas inlet and also to the gas outlet, so that—when the coupling device is accommodated by the first receptacle—breathing gas can flow from the exit opening via the gas inlet into the breathing gas chamber and from the breathing gas chamber via the gas outlet into an environment of the base body.

Such a holder allows easy mounting and/or disassembly of the gas sensor together with the base body.

The first receptacle may be configured to accommodate at least a section of the coupling device, in particular at least a section having the exit opening, with the result that the base body is connected to the coupling device in an airtight manner.

The second receptacle may be configured to accommodate at least a section of the gas sensor in such a way that the base body is connected in an airtight manner to the gas sensor. Additionally or alternatively, the second receptacle may be configured to fix the gas sensor in a defined position and/or orientation relative to the base body. In particular, the holder may be configured such that the gas sensor accommodated by the second receptacle is carried exclusively or largely by the base body in the operational state of the ventilator.

For example, the attachment section may comprise one or more holes for passing one or more screws through it/them. This means that the base body can be easily and quickly screwed onto and/or into the housing. It is possible that the attachment section comprises one or more eyelets and/or threaded receptacles for screwing the base body to the housing. Alternatively or additionally, the attachment section may comprise one or more locking elements such as hooks or lugs for snapping the base body into the housing.

For example, the base body may be made from one-piece and/or made of the same material. In particular, the base body may be an injection molding part, preferably made of plastic.

A second aspect of the invention relates to a ventilator. The ventilator comprises: a breathing gas connector for connecting a breathing gas source to the ventilator; a patient connector for connecting a patient interface to the ventilator; a gas sensor for acquiring a gas concentration; a coupling device comprising a first entrance opening connected to the breathing gas connector, a second entrance opening connected to the patient connector, and an exit opening; wherein the coupling device is adjustable between a calibration position and a measurement position and is configured to fluidically couple the exit opening in the calibration position only to the first entrance opening and in the measurement position either only to the second entrance opening or to both the first entrance opening and the second entrance opening; a housing which surrounds at least a part of the components of the ventilator, wherein the components of the ventilator surrounded by the housing comprise the gas sensor and/or the coupling device; a holder, as described above and below, wherein the base body is fixed via the attachment section on and/or in the housing, wherein the coupling device is accommodated by the first receptacle such that the exit opening is fluidically coupled to the gas inlet, and wherein the gas sensor is accommodated by the second receptacle such that the base body together with the gas sensor accommodated by the second receptacle delimits the breathing gas chamber.

The ventilator can be suitable for invasive and/or non-invasive ventilation of a patient.

For example, a “breathing gas source” can be understood to mean a gas cylinder, a breathing gas mixer, a breathing gas line system (e.g. in a hospital building), or a combination of at least two of these examples.

For example, “patient interface” can be understood to mean a ventilation mask, a tube, or a nasal cannula.

For example, “gas sensor” can be understood to mean an oxygen sensor with a galvanic cell or a paramagnetic oxygen sensor. An optical gas sensor and/or a gas sensor for acquiring a concentration of at least one gas other than oxygen, for example nitrogen, carbon dioxide or anesthetic gas, is also possible.

The gas sensor may thus be arranged on the base body in such a way that the breathing gas passes through the gas sensor as it flows through the breathing gas chamber, with the result that the gas sensor can acquire a gas concentration of the breathing gas passing through the gas sensor.

The coupling device may comprise, for example, a T-shaped or Y-shaped pipe piece. In this case, the exit opening, the first entrance opening and the second entrance opening may be each formed by one of three open ends of the pipe piece.

In addition or alternatively, the coupling device may be formed with a connecting piece having the exit opening (see also further below). The connecting piece may be insertable in an insertion direction into a socket of the second receptacle of the base body in order to fluidically couple the exit opening to the gas inlet of the base body. The connecting piece may be suitably configured such that it is connected to the socket in an airtight manner and/or only with a force-fit when it is inserted into the socket. The exit opening may, for example, be a free, open end of the connecting piece. Optionally, the connecting piece may comprise one or more sealing rings for making an airtight connection to the socket. For example, the sealing ring may extend, viewed in its longitudinal direction, or the sealing rings may extend, viewed in their respective longitudinal directions, in a circumferential direction of an outer and/or inner lateral surface of the connecting piece.

The coupling device may be fixed, for example, screwed, inside the ventilator. Thus, the coupling device remains in the ventilator when the holder, if necessary together with the gas sensor, is removed.

It is possible that the gas sensor is fixed, for example is screwed, to the base body, in particular in such a way that the gas sensor is carried exclusively or largely by the base body.

For example, the gas sensor may be connected via a plug connection to a control apparatus for controlling the operation of the ventilator for data communication and/or power supply.

Various embodiments of the invention are described hereinbelow. These embodiments should not be understood as restricting the scope of the invention.

According to one embodiment, the first receptacle and the coupling device can be connectable to each other to form an (airtight) plug connection. The plug connection can be expediently configured to be releasable, for example by pulling apart the first receptacle and the coupling device with a defined force. For example, the first receptacle may be formed with a socket, wherein the coupling device may be formed with a connecting piece which is complementary to the socket and has the exit opening. Alternatively or additionally, the first receptacle may be formed with a connecting piece, wherein the coupling device may be formed with a socket which is complementary to the connecting piece and has the exit opening. In both cases, the (airtight) plug connection can be established by inserting the respective connecting piece into the respective socket. The term “connecting piece” can generally be understood as a plug that can be inserted into a socket.

According to one embodiment, the first receptacle may comprise a socket for inserting a connecting piece of the coupling device having the exit opening in an insertion direction. Alternatively, or additionally, the first receptacle may comprise a connecting piece for insertion into a socket, which has the exit opening, of the coupling device in an insertion direction. This makes it possible to connect the base body to the coupling device in an airtight manner when it is inserted into the housing without the need to perform additional actions.

According to one embodiment, the socket may have a cross section which tapers in the insertion direction. In this way, a sufficiently force-fitting and permanently airtight connection between the socket and the connecting piece can be established by simply inserting it in the insertion direction. The connection can be released again correspondingly easily when removing the base body by pulling apart the socket and the connecting piece with a specific amount of force. Optionally, the socket may comprise one or more sealing rings for making an airtight connection to the connecting piece. For example, the sealing ring may extend, viewed in its longitudinal direction, or the sealing rings may extend, viewed in their respective longitudinal directions, in a circumferential direction of an outer and/or inner lateral surface of the socket.

According to one embodiment, the second receptacle may comprise a thread for screwing the gas sensor to the base body. Alternatively, the second receptacle may comprise at least three threads for screwing the gas sensor to the base body. This enables a secure and easy-to-detach fixation of the gas sensor to the base body.

According to one embodiment, the threads may comprise a central thread and at least two screw threads arranged around the central thread, wherein a screw can be screwed into each of the screw threads. Additionally or alternatively, the central thread may be formed to accommodate a housing thread on a cylindrical section of a housing of the gas sensor, in particular an outer housing of the gas sensor. For example, the threads may be arranged such that their center axes (which are, for example, parallel to each other) intersect a common straight line. The central thread may lie between the two screw threads. However, any other arrangements are also conceivable. It is possible that the central thread has a much larger diameter than each of the screw threads and/or delimits the breathing gas chamber. In other words, at least a section of the central thread may be a section of the base body delimiting the breathing gas chamber. Additionally or alternatively, the housing thread can or the cylindrical section can, or the housing thread and the cylindrical section can, delimit the breathing gas chamber. In other words, at least a section of the housing thread and/or of the cylindrical section may be a section of the gas sensor delimiting the breathing gas chamber. For example, the diameter of the central thread may be at least 2 times, at least 3 times, or at least 4 times the diameter of each of the screw threads.

According to one embodiment, the base body may further comprise a connecting channel. In this case, the gas inlet may be connected to the breathing gas chamber via the connecting channel. Additionally, a constriction may be arranged between the connecting channel and the breathing gas chamber. The constriction may be configured such that a flow of the breathing gas entering the breathing gas chamber via the constriction is in a desired value range depending on a pressure of the breathing gas in the connecting channel. The constriction may be formed with a (significantly) smaller flow cross section than the connecting channel. For example, the flow cross section of the constriction may be at most about 90%, at most about 50%, at most about 10% or at most about 5% of the flow cross section of the connecting channel. The constriction may, for example, be formed as a nozzle upstream of the breathing gas chamber.

According to one embodiment, the second receptacle may comprise a mountable adapter for attaching the gas sensor to the base body, with the adapter being separate from the remaining base body. For example, the adapter can be screwable to the remaining base body and/or the gas sensor by means of a plurality of screws. The adapter may also be formed to be part of a housing and/or a bearing structure of the gas sensor. For example, the adapter may be a frame-type and/or plate-type adapter. This makes it possible to optionally attach different types of gas sensors to the base body without having to specifically adapt the base body or the second receptacle to the respective type.

According to one embodiment, the base body may form a part of an outer wall and/or inner wall of the housing when it is attached to and/or in the housing.

According to one embodiment, the housing may have a (continuous) housing opening for inserting the gas sensor into the interior of the ventilator. In this case, the base body can partially or completely cover the housing opening when it is attached to and/or in the housing.

According to one embodiment, the first receptacle and the second receptacle may be arranged on a first side of the base body facing the interior of the housing (or of the ventilator). In this case, a second side of the base body opposite the first side may form a part of the outer wall of the housing.

According to one embodiment, the coupling device can be adjustable between the calibration position and the measurement position depending on a pressure difference between a first breathing gas pressure applied to the first entrance opening and a second breathing gas pressure applied to the second entrance opening, for example an exhalation pressure. For example, the coupling device can be adjustable only depending on the pressure.

According to one embodiment, the coupling device may be in the calibration position when the first breathing gas pressure is greater than the second breathing gas pressure or equal to the second breathing gas pressure. Additionally or alternatively, the coupling device may be in the measurement position if the first breathing gas pressure is less than the second breathing gas pressure. In other words, it is possible that the coupling device is in the calibration position if the pressure difference is not equal to zero and has a first sign (for example, positive) or if the pressure difference is equal to zero, and is in the measurement position if the pressure difference is non-zero and has a second sign opposite to the first sign (for example, negative).

According to one embodiment, the coupling device may further comprise a base and a sealing element which is connected to the base and is deflectable relative to the base. In this case, the sealing element can be positioned by means of the base in a flow path of the breathing gas through the coupling device such that the sealing element is deflectable relative to the base between the calibration position and the measurement position depending on the pressure difference between the first breathing gas pressure and the second breathing gas pressure. The sealing element can seal the second entrance opening in an airtight manner in the calibration position and release it in the measurement position. For example, the calibration position can correspond to a rest position of the sealing element. Alternatively, the measurement position can correspond to the rest position. The term “base” can generally be used to refer to a component part for supporting the sealing element so it is held and/or can move. “Sealing element” can be understood to mean a membrane or piston, for example. The base and the sealing element can be formed from multiple parts or from one part. For example, the base and the sealing element in a one-piece design can be made of the same material (e.g. plastic), in particular as an injection molding part.

According to one embodiment, the sealing element may be formed in such a resilient manner and/or be connected to the base in such a resilient manner that, in the measurement position, it is subjected to a restoring force acting in the direction of the calibration position. This allows the sealing element to return to the calibration position on its own, unless it is otherwise prevented from doing so. The sealing element may be configured such that it allows a flow of the breathing gas from the first entrance opening to the exit opening either only in the calibration position or in both the calibration position and the measurement position.

According to one embodiment, the sealing element can be elastically resiliently connected to the base via at least two elastically deformable spring arms. The base, the sealing element and the spring arms can be formed from multiple parts or from one part. For example, the base, the sealing element and the spring arms in a one-piece design can be made of the same material (e.g. plastic), in particular as an injection molding part.

According to one embodiment, each of the spring arms may comprise two shorter longitudinal sections and one longer longitudinal section connecting the two shorter longitudinal sections. The longer longitudinal section may be longer than either of the two shorter longitudinal sections or longer than the two shorter longitudinal sections combined. A first of the two shorter longitudinal sections may protrude from the longer longitudinal section, for example from its first end, in a first direction deviating from a longitudinal direction of the longer longitudinal section, whereas a second of the two shorter longitudinal sections can protrude from the longer longitudinal section, for example from its second end, in a second direction deviating from the first direction and/or the longitudinal direction of the longer longitudinal section. For example, the first and second directions may be opposite directions, orthogonal directions relative to each other, or oblique directions relative to each other. For example, the longer longitudinal section may be at least 2 times, at least 4 times, or at least 10 times as long as either of the two shorter longitudinal sections or the two shorter longitudinal sections combined. Each of the spring arms can be connected to the base via its first shorter longitudinal section and to the sealing element via its second shorter longitudinal section.

According to one embodiment, the longer longitudinal section can be at least partially curved viewed in its longitudinal direction. Such spring arms can make the suspension more efficient and/or more durable in a given installation space compared to variants with differently shaped spring arms.

According to one embodiment, the base may be ring-shaped and/or the sealing element may be disk-shaped. In addition, the ring-shaped base and the disk-shaped sealing element can have a common center axis, i.e. be arranged concentrically to each other. In this case, the sealing element can be arranged opposite and/or within an opening in the base and/or can be deflectable along the common center axis relative to the base between the calibration position and the measurement position.

### BRIEF DESCRIPTION OF THE DRAWINGS

Embodiments of the invention are described hereinafter with reference to the accompanying drawings. Neither the description nor the drawings should be understood as restricting the scope of the invention. In the drawings:

FIG. 1 shows a ventilator according to an embodiment of the invention.

FIG. 2 shows a holder according to an embodiment of the invention, the base body of which is screwed to a gas sensor in the form of a paramagnetic measurement cell.

FIG. 3 shows the holder of FIG. 2 without the gas sensor and with a coupling device inserted.

FIG. 4 shows a gas sensor in the form of a chemical measurement cell, the housing of which can be screwed to the base body of the holder from FIG. 3.

FIG. 5 shows a cross section through the coupling device from FIG. 3.

FIG. 6 shows a cross section through the base body of FIG. 3 and the gas sensor from FIG. 4 which is screwed thereto.

FIG. 7 shows a top view of a sealing membrane for use in a coupling device of a ventilator according to an embodiment of the invention.

The drawings are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

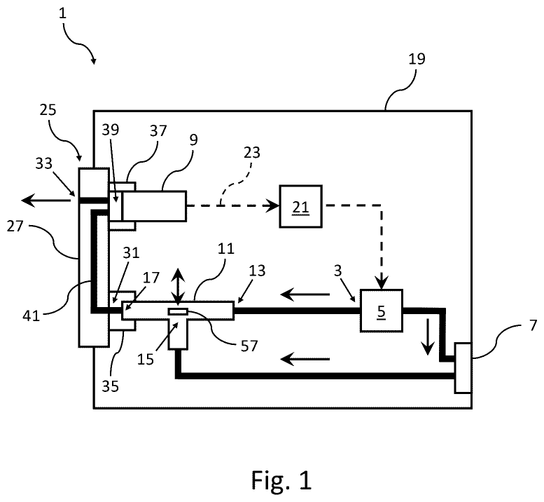

FIG. 1 shows components of a ventilator 1. In this example, the ventilator 1 comprises a breathing gas connector 3 for connecting a breathing gas source 5 to the ventilator 1, a patient connector 7 for connecting a patient interface to the ventilator 1, a gas sensor 9 for acquiring a gas concentration, and a coupling device 11. The coupling device 11 has a first entrance opening 13 connected to the breathing gas connector 3, a second entrance opening 15 connected to the patient connector 7, and an exit opening 17. In addition, the coupling device 11 is adjustable between a calibration position and a measurement position and formed to fluidically couple the exit opening 17 in the calibration position only to the first entrance opening 13 and in the measurement position either only to the second entrance opening 15 or both to the first entrance opening 13 and to the second entrance opening 15, with the result that breathing gas can flow in the calibration position from the first entrance opening 13 and thus from the breathing gas source 5 (for example, a gas cylinder, a breathing gas mixer or a breathing gas line system) to the exit opening 17 and can flow in the measurement position at least from the second entrance opening 15 and thus at least from the patient interface (for example, a breathing mask, a tube or a nasal cannula) to the exit opening 17.

Further, the ventilator 1 comprises a housing 19, which surrounds at least a part of the components of the ventilator 1, in this case, among other things, the breathing gas source 5, the gas sensor 9, the coupling device 11 and an optional control apparatus 21 for controlling the breathing gas source 5 using a sensor signal 23 provided by the gas sensor 9.

In addition, the ventilator 1 comprises a holder 25, via which the gas sensor 9 is attached, for example screwed, to the housing 19, in particular in such a way that the gas sensor 9 is completely or largely located inside the housing 19.

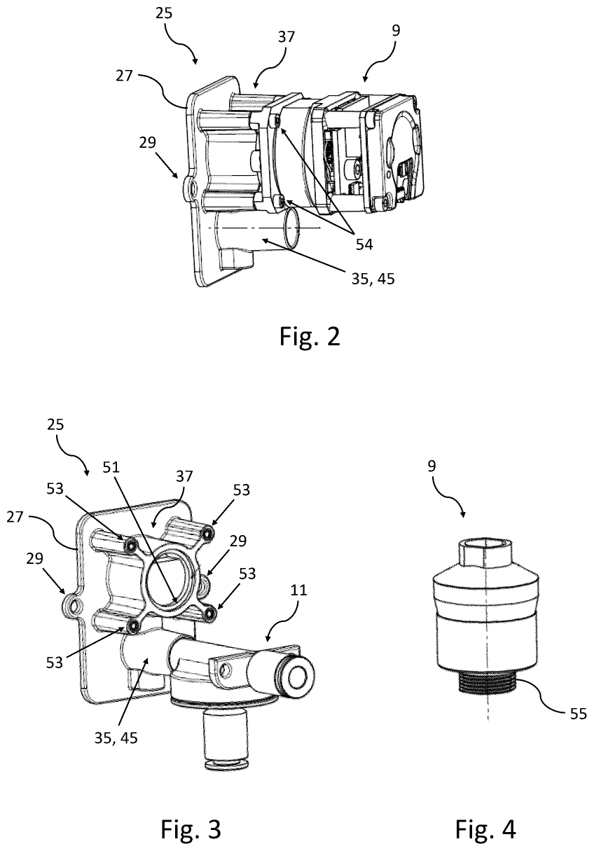

The holder 25 comprises a base body 27 with an attachment section 29 (see FIG. 2 and FIG. 3), via which the base body 27 is attached, here screwed, to the housing 19, a gas inlet 31, a gas outlet 33, a first receptacle 35 and a second receptacle 37. In this example, the two receptacles 35, 37 are arranged on the base body 27 in such a way that they lie one above the other in the operational state of the ventilator 1, as shown in FIG. 1. As an alternative or in addition to different vertical positions, the two receptacles 35, 37 may have different horizontal positions when the ventilator 1 is operational.

The first receptacle 35 is configured to accommodate least a section of the coupling device 11, so that the exit opening 17 is fluidically coupled to the gas inlet 31.

The second receptacle 37 is configured to accommodate at least a section of the gas sensor 9. The base body 27, together with the gas sensor 9 accommodated by the second receptacle 37, delimits a breathing gas chamber 39, which is connected first to the gas inlet 31 and to the gas outlet 33 on the other hand, such that the breathing gas can flow from the exit opening 17 via the gas inlet 31 into the breathing gas chamber 39 and from there via the gas outlet 33 into an environment of the base body 27. When flowing through the breathing gas chamber 39, the breathing gas passes through the gas sensor 9, such that the gas sensor 9 can acquire the gas concentration of the breathing gas passing through the gas sensor 9 and generate a corresponding sensor signal 23.

A flow direction of the breathing gas through the ventilator 1 is indicated in FIG. 1 by means of continuous arrows by way of example.

The housing 19 may have a separate housing opening for inserting the gas sensor 9 attached to the holder 25 into the interior of the housing 19 (or into the interior of the ventilator 1). The housing opening can be advantageously formed in a side wall of the housing 19. However, other positions of the housing opening, for example in a bottom or top of the ventilator 1 in the operational state, are also possible. The base body 27 can cover the housing opening in the mounted state of the holder 25 partially or completely, in particular to be dust-proof and/or fluid-tight. In this case, the base body 27 can form a part of an outer and/or inner surface of the housing 19.

The two receptacles 35, 37 may, in the mounted state of the holder 25, be located on a first side of the base body 27 facing the interior of the housing 19 (or the interior of the ventilator 1). In this case, a second side of the base body 27 opposite the first side can form a part of the outer surface or an outer wall of the housing 19.

In addition, the base body 27 may comprise a connecting channel 41, which is connected at its first end to the gas inlet 31 and at its second end to the breathing gas chamber 39, so that the breathing gas can flow via the connecting channel 41 from the gas inlet 31 to the breathing gas chamber 39. The connecting channel 41 can be formed, for example, by a corresponding cutout in a material of the base body 27.

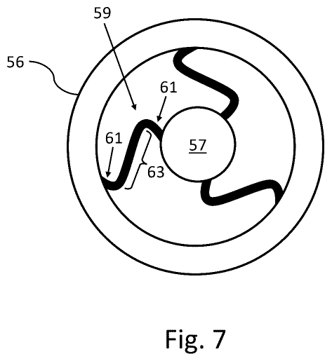

As shown in FIG. 6, optionally a constriction 43 with a significantly smaller flow cross section than the connecting channel 41 can be arranged between the second end of the connecting channel 41 and the breathing gas chamber 39. For example, the constriction 43 can act as a kind of nozzle.

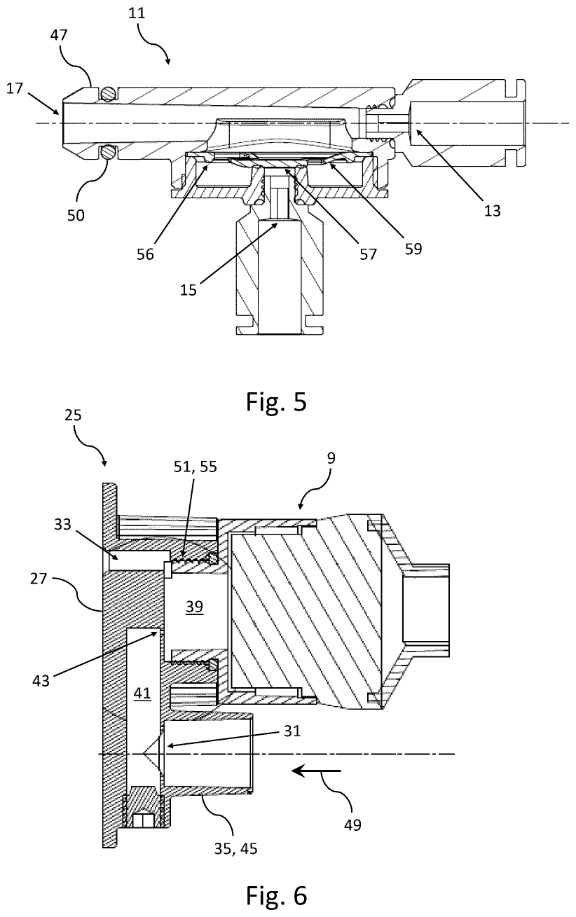

In FIG. 2, FIG. 3 and FIG. 6, an optional embodiment of the first receptacle 35 in the form of a socket 45 is also shown. Into the socket 45, a connecting piece 47 of the coupling device 11 (see FIG. 5) having the exit opening 17 can be inserted in an insertion direction 49 (see FIG. 6). Such a plug-and-socket system enables an uncomplicated mechanical and fluidic coupling of the coupling device 11 to the base body 27, in particular in such a way that the coupling device 11 is airtight and/or is exclusively connected to the base body 27 in a force-fitting manner.

As can be seen in FIG. 6, the socket 45 may have a cross section slightly tapered in the insertion direction 49. This has the effect that the socket 45 or the connecting piece 47 is subjected to a radial force when inserting the connecting piece 47 into the socket 45, which force is the greater the deeper the connecting piece 47 is inserted into the socket 45. In this way, sufficient tightness can be ensured even with very little force application in the insertion direction 49 and also in the event of major deviations in dimensions and/or shape due to assembly, production and/or temperature, without additional measures being necessary. The connection can also be released correspondingly easily.

Depending on the embodiment, at least one sealing ring can additionally extend around an inner and/or outer lateral surface of the socket 45 and/or the connecting piece 47. The sealing ring or the sealing rings may be slightly compressed when inserting the connecting piece 47 into the socket 45 to provide an airtight connection between the socket 45 and the connecting piece 47.

In the example shown in FIG. 5, such a sealing ring 50 extends around an outer lateral surface of the connecting piece 47.

As can be seen in FIG. 3, the second receptacle 37 may comprise a plurality of threads for screwing the base body 27 to different types of the gas sensor 9. In this example, the second receptacle 37 comprises a central thread 51 for screwing the base body 27 to a galvanic oxygen sensor and four screw threads 53 for screwing the base body 27 to a paramagnetic oxygen sensor (as an alternative to the galvanic oxygen sensor) via four separate screws 54 (see FIG. 2). The screw threads 53 can be distributed evenly around the central thread 51. The threads 51, 53 can be configured as internal threads, as shown here. However, external threads are also possible.

The central thread 51 can have a much larger thread diameter than any of the screw threads 53. In particular, the central thread 51 can be screwed by a housing thread 55 to a cylindrical section of a housing of the gas sensor 9 in the form of the galvanic oxygen sensor (see FIG. 4).

FIG. 2 shows the base body 27 and the gas sensor 9, screwed thereto by means of a plurality of screws 54, in the form of the paramagnetic oxygen sensor in a perspective view.

FIG. 6 shows the base body 27 and the gas sensor 9 screwed thereto in the form of the galvanic oxygen sensor from FIG. 4 in cross section. As can be seen here, the cylindrical section, having the housing thread 55, of the housing of the gas sensor 9, more precisely its inner lateral surface, can form a section of the gas sensor 9 delimiting the breathing gas chamber 39.

FIG. 5 shows a cross section through the coupling device 11 from FIG. 3. The coupling device 11 may be configured such that it is adjusted between the calibration position and the measurement position depending on a pressure difference between a first breathing gas pressure applied to the first entrance opening 13 and a second breathing gas pressure applied to the second entrance opening 15. For example, the coupling device 11 may be in the calibration position when the first breathing gas pressure is greater than the second breathing gas pressure or equal to the second breathing gas pressure, and in the measurement position when the first breathing gas pressure is less than the second breathing gas pressure.

In order to enable such a pressure-dependent adjustment, the coupling device 11 may comprise a special sealing membrane.

FIG. 7 shows a possible embodiment of the sealing membrane in a top view. The sealing membrane comprises a base 56 and a sealing element 57 which is connected to the base 56 and is deflectable relative to the base 56. The sealing element 57 can be positioned by means of the base 56 in a flow path of the breathing gas through the coupling device 11 such that the sealing element 57 is deflectable relative to the base 56 depending on the aforementioned pressure difference between the calibration position and the measurement position. For example, the calibration position can correspond to a rest position of the sealing element 57, in which the sealing element 57 closes the second entrance opening 15 in an airtight manner and thus blocks a flow path of the breathing gas between the patient connector 7 and the exit opening 17. By deflecting the sealing element 57 into the measurement position, for example due to the breathing gas pressure generated during exhalation, the second entrance opening 15 can be released.

Conveniently, the sealing element 57 can be formed in such a resilient manner and/or be connected to the base 56 in such a resilient manner that, in the measurement position, it is subjected to a restoring force acting in the direction of the calibration position.

As can be seen in FIG. 7, the sealing element 57 can be formed for example in a disk shape and connected to the base 56 elastically by means of a plurality of, here three, elastically deformable spring arms 59 in order to allow the deflection between the calibration position and the measurement position. In this case, the base 56 may be ring-shaped and arranged concentrically to the sealing element 57. The sealing element 57 can accordingly be deflectable in the direction of a common center axis of the base 56 and of the sealing element 57 relative to the base 56.

It is possible that each of the spring arms 59 comprises two shorter longitudinal sections 61 and one longer longitudinal section 63 which connects the two shorter longitudinal sections 61 and may be significantly longer than either of the two shorter longitudinal sections 61 or than the two shorter longitudinal sections 61 together.

In the example shown in FIG. 7, each of the spring arms 59 is connected via its two shorter longitudinal sections 61 to an inner edge of the base 56 on the one hand and to an outer edge of the sealing element 57 on the other hand.

The two shorter longitudinal sections 61 may protrude in different, for example opposite, directions from the ends of the respective longer longitudinal section 63, wherein these directions may deviate from a longitudinal direction of the respective longer longitudinal section 63 in each case.

For example, the longer longitudinal sections 63 can be at least partially curved when viewed in their respective longitudinal direction. This makes it possible to design the spring arms 59—at a given distance of the outer edge of the sealing element 57 from the inner edge of the base 56 in the radial or axial direction—to be as long as possible and correspondingly flexible.

Finally, it will be noted that terms such as “have”, “comprise”, “include”, “with” etc. do not exclude other elements or steps, and indefinite articles such as “a” or “an” do not exclude a plurality.

Furthermore, it is pointed out that features or steps described with reference to one of the embodiments above can also be used in combination with features or steps described with reference to other embodiments from among the embodiments above.

List of reference numerals

| | |
|---|---|
| 1 | Ventilator |
| 3 | Breathing gas connector |
| 5 | Breathing gas source |
| 7 | Patient connector |
| 9 | Gas sensor |
| 11 | Coupling device |
| 13 | First entrance opening |
| 15 | Second entrance opening |
| 17 | Exit opening |
| 19 | Housing |
| 21 | Control apparatus |
| 23 | Sensor signal |
| 25 | Holder |
| 27 | Base body |
| 29 | Attachment section |
| 31 | Gas inlet |
| 33 | Gas outlet |
| 35 | First receptacle |
| 37 | Second receptacle |
| 39 | Breathing gas chamber |
| 41 | Connecting channel |
| 43 | Constriction |
| 45 | Socket |
| 47 | Connecting piece |
| 49 | Insertion direction |
| 50 | Sealing ring |
| 51 | Central thread |
| 53 | Screw thread |
| 54 | Screw |
| 55 | Housing thread |
| 56 | Base |
| 57 | Sealing element |
| 59 | Spring arm |
| 61 | Shorter longitudinal section |
| 63 | Longer longitudinal section |

What is claimed is:

1. A holder for attaching a gas sensor to and/or in a housing of a ventilator which comprises, in addition to the gas sensor and the housing, a breathing gas connector for connecting a breathing gas source, a patient connector for connecting a patient interface, and a coupling device for fluidically coupling the gas sensor to the breathing gas connector and/or the patient connector, wherein the holder comprises a base body having an attachment section for attachment of the base body to and/or in the housing, a gas inlet, a gas outlet, a first receptacle for accommodating the coupling device such that an exit opening of the coupling device is fluidically coupled to the gas inlet, and a second receptacle for accommodating the gas sensor, the base body being configured to delimit, together with the gas sensor accommodated by the second receptacle, a breathing gas chamber which is connected to the gas inlet on the one hand and to the gas outlet on the other hand such that—when the coupling device is accommodated by the first receptacle—breathing gas can flow from the exit opening via the gas inlet into the breathing gas chamber and from the breathing gas chamber via the gas outlet into an environment of the base body.

2. The holder of claim 1, wherein the first receptacle comprises a socket for inserting a connecting piece of the coupling device comprising the exit opening in an insertion direction.

3. The holder of claim 2, wherein the socket has a cross section which tapers in the insertion direction.

4. The holder of claim 1, wherein the second receptacle comprises a thread for screwing the gas sensor to the base body.

5. The holder of claim 1, wherein the second receptacle comprises at least three threads for screwing the gas sensor to the base body.

6. The holder of claim 5, wherein the at least three threads comprise a central thread and at least two screw threads arranged around the central thread, the central thread being configured to accommodate a housing thread on a cylindrical section of a housing of the gas sensor, wherein one screw can be screwed into each of the screw threads arranged around the central thread.

7. The holder of claim 1, wherein the base body further comprises a connecting channel and the gas inlet is connected via the connecting channel to the breathing gas chamber, a constriction with a reduced flow cross section compared to the connecting channel being arranged between the connecting channel and the breathing gas chamber.

8. A ventilator, wherein the ventilator comprises:
a breathing gas connector for connecting a breathing gas source;
a patient connector for connecting a patient interface;
a gas sensor for acquiring a gas concentration;
a coupling device comprising a first entrance opening connected to the breathing gas connector, a second entrance opening connected to the patient connector, and an exit opening, the coupling device being adjustable between a calibration position and a measurement position and being configured to fluidically couple the exit opening in the calibration position only to the first entrance opening and in the measurement position either only to the second entrance opening or to both the first entrance opening and the second entrance opening;
a housing which surrounds at least a part of components of the ventilator, the components of the ventilator surrounded by the housing comprising the gas sensor and/or the coupling device;
the holder of claim 1,
wherein the base body is attached via the attachment section to and/or in the housing and the coupling device is accommodated by the first receptacle and the gas sensor is accommodated by the second receptacle.

9. The ventilator of claim 8, wherein the base body forms a part of an outer wall and/or an inner wall of the housing and/or the housing has a housing opening for inserting the gas sensor and the base body covers the housing opening at least partially.

10. The ventilator of claim 9, wherein the first receptacle and the second receptacle are arranged on a first side of the base body facing an interior of the housing and a second side of the base body opposite the first side forms a part of an outer wall of the housing.

11. The ventilator of claim 8, wherein the coupling device is adjustable between a calibration position and a measurement position depending on a pressure difference between a first breathing gas pressure applied to the first entrance opening and a second breathing gas pressure applied to the second entrance opening.

12. The ventilator of claim 11, where the coupling device is in the calibration position when the first breathing gas pressure is greater than the second breathing gas pressure or equal to the second breathing gas pressure, and/or is in the measurement position when the first breathing gas pressure is less than the second breathing gas pressure.

13. The ventilator of claim 11, wherein the coupling device further comprises a base and a sealing element which is connected to the base and is deflectable relative to the base, the sealing element being positioned in a flow path of the breathing gas through the coupling device by means of the base in such a way that the sealing element is deflectable between the calibration position and the measurement position relative to the base depending on a pressure difference between the first breathing gas pressure and the second breathing gas pressure, and wherein the sealing element seals the second entrance opening in the calibration position in an airtight manner and releases it in the measurement position.

14. The ventilator of claim 13, wherein the sealing element is configured to be resilient and/or is connected to the base in such a resilient manner that it is subjected in the measurement position to a restoring force acting in a direction of the calibration position; and/or is resiliently connected to the base via at least two elastically deformable spring arms.

15. The ventilator of claim 14, wherein each of the spring arms comprises two shorter longitudinal sections and one longer longitudinal section connecting the two shorter longitudinal sections, the longer longitudinal section being longer than either of the two shorter longitudinal sections or longer than the two shorter longitudinal sections combined, wherein a first of the two shorter longitudinal sections protrudes from a first end of the longer longitudinal section in a first direction deviating from a longitudinal direction of the longer longitudinal section and a second of the two shorter longitudinal sections protrudes from a second end of the longer longitudinal section in a second direction deviating from the first direction and/or the longitudinal direction of the longer longitudinal section.

16. The ventilator of claim 13, wherein the base is ring-shaped and/or the sealing element is disk-shaped.
